SEQUENCE LISTING (2 sequences)

SEQ ID NO: 1 - protein, 121 aa, Homo sapiens
MAPRPLLLLLLLLGGSAARPAPPRARRHSDGTFTSELSRLREGARLQRLLQGLVGKRSEQDAENSMAWTRLSAGLLCPSGSNMPILQAWMPLDGTWSPWLPPGPMVSEPAGAAAEGTLRPR

SEQ ID NO: 2 - protein, 27 aa, Homo sapiens
HSDGTFTSELSRLREGARLQRLLQGLV

(19) United States
(12) Patent Application Publication
(10) Pub. No.: US 2019/0365863 A1
(43) Pub. Date: Dec. 5, 2019

(54) METHODS AND MATERIALS FOR TREATING FUNCTIONAL DYSPEPSIA

(71) Applicant: Mayo Foundation for Medical Education and Research, Rochester, MN (US)

(72) Inventor: Laurence J. Miller, Scottsdale, AZ (US)

(73) Assignee: Mayo Foundation for Medical Education and Research, Rochester, MN (US)

(21) Appl. No.: 16/470,815

(22) PCT Filed: Dec. 20, 2017
(86) PCT No.: PCT/US2017/067519

§ 371 Date: Jun. 18, 2019

Related U.S. Application Data
(60) Provisional application No. 62/436,871, filed on Dec. 20, 2016.

(51) Int. Cl. A61K 38/22; A61K 31/225

(52) U.S. Cl. CPC A61K 38/2235; A61K 45/06; A61K 31/225

(57) ABSTRACT

This document relates to methods and materials for treating functional dyspepsia. For example, methods and materials for using secretin to treat functional dyspepsia are provided.

### CROSS-REFERENCE TO RELATED APPLICATIONS

This application claims priority to U.S. Application Ser. No. 62/436,871, filed on Dec. 20, 2016. The disclosure of the prior application is considered part of the disclosure of this application, and is incorporated in its entirety into this application.

### BACKGROUND

### 1. Technical Field

This document relates to methods and materials for treating functional dyspepsia. For example, this document provides methods and materials for using secretin, a secretin receptor agonist, a positive allosteric modulator, or a degrading enzyme inhibitor such as a neprilysin inhibitor that increases bioactive secretin to treat functional dyspepsia.

### 2. Background Information

Functional dyspepsia (FD) is a common clinical condition associated with a complex of upper abdominal symptoms including: upper centered discomfort and pain, feeling of abdominal fullness, early satiety, abdominal distention and bloating, recurrent retching and/or vomiting and nausea. The exact prevalence of dyspepsia in the general population is not known, but it is estimated that one in ten people suffer from FD. Patients with chronic or recurrent dyspeptic symptoms frequently undergo repeated, expensive, and invasive tests such as endoscopy. Current medical treatment includes eradication of H. pylori, acid suppression, prokinetic drugs, antidepressants, and psychological and alternative therapy. Despite these treatments, many patients remain refractory to current therapies with continued disabling symptoms. FD impairs quality of life, work performance, and family relationships and incurs a high healthcare cost worldwide.

### SUMMARY

This document provides methods and materials for treating FD. For example, this document provides methods and materials for administering a composition that includes secretin, a secretin receptor agonist, or positive allosteric modulator to a mammal having FD to treat FD. In some cases, inhibition of secretin degradation using a protease inhibitor, such as a neprilysin inhibitor, can be used in addition to secretin or a secretin agonist or in place of secretin or a secretin agonist to treat FD.

As described herein, secretin biosynthesis and secretion can occur in enteroendocrine S cells in the duodenum, site of a pathologic response in FD. Secretin can stimulate gastric accommodation through relaxation of the gastric fundus, a process that is defective in FD. Secretin can achieve this through stimulation of secretin receptors on vagal afferent neurons, with this activity possibly mediated through release of vasoactive intestinal polypeptide (VIP) and prostaglandins. This relaxation of the gastric fundus can increase gastric accommodation and reduce abdominal pain, thereby providing relief to mammals suffering from FD.

In general, this document features a method for treating functional dyspepsia in a mammal. The method comprises, or consists essentially of, administering a composition comprising secretin to a mammal identified as having functional dyspepsia, wherein a symptom of the functional dyspepsia is reduced. The mammal can be a human. The secretin can be human secretin. The sole active ingredient of the composition can be secretin. The composition can comprise a positive allosteric modulator of secretin. The composition can comprise a neprilysin inhibitor. The neprilysin inhibitor can be sacubitril or sacubitrilat. The composition can be administered intravenously or orally to the mammal.

In another aspect, this document features a method for treating functional dyspepsia in a mammal. The method comprises, or consists essentially of, orally administering a composition comprising secretin to a mammal identified as having functional dyspepsia, wherein a symptom of the functional dyspepsia is reduced. The mammal can be a human. The secretin can be human secretin. The sole active ingredient of the composition can be secretin.

In another aspect, this document features a method for treating functional dyspepsia in a mammal. The method comprises, or consists essentially of, administering a composition comprising neprilysin inhibitor to a mammal identified as having functional dyspepsia, wherein a symptom of the functional dyspepsia is reduced. The mammal can be a human. The neprilysin inhibitor can be sacubitril or sacubitrilat. The sole active ingredient of the composition can be the neprilysin inhibitor. The composition can comprise secretin. The secretin can be a human secretin. The composition can comprise a positive allosteric modulator of secretin. The composition can be administered intravenously or orally to the mammal.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DETAILED DESCRIPTION

This document provides methods and materials for treating FD. For example, this document provides methods and materials for administering a composition that includes secretin to a mammal having FD to treat FD. In some cases, secretin can be administered to a mammal (e.g., a human with FD) to reduce the symptoms of FD. Examples of symptoms of FD that can be treated as described herein include, without limitation, upper centered discomfort and pain, feeling of abdominal fullness, early satiety, abdominal distention and bloating, recurrent retching, vomiting, nausea, and combinations thereof. In some cases, a composition that includes secretin can be administered to a mammal to treat FD and/or to reduce the symptoms of FD with secretin being the sole active ingredient.

As described herein, secretin is a hormone that is synthesized and secreted by enteroendocrine S cells in the duodenum that relaxes the gastric fundus (increases gastric accommodation) through its action on vagal afferent neurons. Contributing to this effect is the release of VIP and prostaglandins. This relaxation of the gastric fundus can increase gastric accommodation and reduce abdominal pain from FD.

Any appropriate secretin can be used as described herein. For example, a human, monkey, bovine, horse, pig, dog, cat, mouse, or rat secretin can be used as described herein to treat FD and/or to reduce the symptoms of FD. In some cases, a human secretin having the amino acid sequence set forth in GenBank Accession No. P09683 (i.e., MAPRPLLLLLLLLGGSAARPAPPRARRHSDGTFTSELSRLR-EGARLQRLLQGLVGKRSEQDAENSMAWTRLSAGLLCPSGSNMPILQAWMPL DGTWSPWLPPGPMVSEPAGAAAEGTLRPR; SEQ ID NO:1) can be used as described herein. In some cases, secretin can be used as described herein in its mature form. For example, a mature form of human secretin can be used as described herein to treat FD and/or to reduce the symptoms of FD. A mature form of human secretin can have the following amino acid sequence: HSDGTFTSELSRLREGARL-QRLLQGLV-amide (SEQ ID NO:2).

In some cases, one or more secretin receptor agonists and/or positive allosteric modulators of secretin (e.g., one, two, three, four, or more secretin receptor agonists and/or positive allosteric modulators of secretin) can be used in addition to secretin or in place of secretin to treat FD and/or to reduce the symptoms of FD as described herein. When using a secretin receptor agonist or a positive allosteric modulator of secretin in place of secretin, a composition that containing the secretin receptor agonist or positive allosteric modulator of secretin can be administered to a mammal to treat FD and/or to reduce the symptoms of FD with the secretin receptor agonist or the positive allosteric modulator of secretin being the sole active ingredient.

In some cases, one or more inhibitors of enzymes known to degrade secretin such as neprilysin inhibitors (e.g., one, two, three, four, or more neprilysin inhibitors) can be used in addition to secretin or in place of secretin to treat FD and/or to reduce the symptoms of FD as described herein. Examples of neprilysin inhibitors include, without limitation, sacubitril and sacubitrilat. When using a degradative enzyme inhibitor (e.g., a neprilysin inhibitor) in place of secretin, a composition that containing the degradative enzyme inhibitor (e.g., neprilysin inhibitor) can be administered to a mammal to treat FD and/or to reduce the symptoms of FD with the degradative enzyme inhibitor (e.g., a neprilysin inhibitor) being the sole active ingredient.

Any appropriate mammal having FD can be treated as described herein. For example, humans and other primates such as monkeys having FD can be treated with secretin. In some cases, dogs, cats, horses, bovine species, pigs, sheep, rabbits, mice, and rats can be treated with secretin as described herein.

Any appropriate method can be used to identify a mammal having FD. For example, upper gastrointestinal endoscopy, gastric accommodation scan, gastric emptying studies, and barium study of the upper gastrointestinal tract can be used to identify a human or other mammal having FD. Once identified as having FD, the mammal can be administered (or instructed to self-administer) a composition containing secretin, one or more positive allosteric modulators of secretin, one or more neprilysin inhibitors, or a combination thereof (e.g., secretin plus a neprilysin inhibitor such as sacubitril or sacubitrilat). For example, a composition containing secretin as the sole active ingredient can be administered to a human identified as having FD to reduce the severity of one or more symptoms of FD. In some cases, one or more acid suppression agents and/or prokinetic agents can be included in a composition containing secretin, one or more secretin receptor agonists, one or more positive allosteric modulators of secretin, one or more neprilysin inhibitors, or a combination thereof to help treat FD and/or help reduce the symptoms of FD. Examples of acid suppression agents that can be administered to a mammal having FD as described herein include, without limitation, H2 receptor blockers such as tagamet and proton pump inhibitors such as omeprazole.

In some cases, secretin, one or more secretin receptor agonists, one or more positive allosteric modulators of secretin, one or more neprilysin inhibitors, or a combination thereof can be formulated into a pharmaceutically acceptable composition for administration to a mammal having FD. For example, a therapeutically effective amount of secretin can be formulated together with one or more pharmaceutically acceptable carriers (additives) and/or diluents. A pharmaceutical composition can be formulated for administration in solid or liquid form including, without limitation, sterile solutions, suspensions, sustained-release formulations, tablets, capsules, pills, powders, and granules. In some cases, secretin can be formulated into a sustained release composition as described elsewhere (see, e.g., U.S. Pat. No. 5,980,945) and administered to a mammal (e.g., a human) to treat FD and/or to reduce the symptoms of FD as described herein.

Pharmaceutically acceptable carriers, fillers, and vehicles that may be used in a pharmaceutical composition described herein include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A pharmaceutical composition containing secretin, one or more secretin receptor agonists, one or more positive allosteric modulators of secretin, one or more neprilysin inhibitors, or a combination thereof can be designed for oral or parenteral (such as subcutaneous, intramuscular, intravenous, or intradermal) administration. In some cases, a composition containing secretin, one or more positive allosteric modulators of secretin, one or more neprilysin inhibitors, or a combination thereof can be formulated for sublingual administration (e.g., drops under the tongue) or for transdermal administration (e.g., for use as a skin patch).

When being administered orally, a pharmaceutical composition containing secretin, one or more secretin receptor agonists, one or more positive allosteric modulators of secretin, one or more neprilysin inhibitors, or a combination thereof can be in the form of a pill, tablet, or capsule. Compositions suitable for parenteral administration include aqueous and non-aqueous sterile injection solutions that can contain anti-oxidants, buffers, bacteriostats, and solutes which render the formulation isotonic with the blood of the intended recipient; and aqueous and non-aqueous sterile suspensions which may include suspending agents and thickening agents.

In some cases, a pharmaceutical composition can be formulated to include secretin (or one or more secretin receptor agonists, one or more positive allosteric modulators of secretin, one or more neprilysin inhibitors, or a combination thereof) for oral administration to a mammal (e.g., a human) having FD. Any appropriate method can be used to formulate secretin into a composition for oral delivery to a mammal (e.g., a human) having FD. For example, the methods described elsewhere (see, e.g., International PCT Patent Application Publication No. WO 2016/120378, International PCT Patent Application Publication No. WO 2016/120380, or U.S. Pat. No. 5,206,219), which describe formulating GLP-1 agonist compositions for oral delivery, can be used to formulate secretin into a composition for oral delivery by replacing the GLP-1 agonist with secretin. In some cases, the formulation and coating techniques described in the section extending from page 2, line 14 to page 13, line 17 of International PCT Patent Application Publication No. WO 2016/120378 or the formulation and coating techniques described in the section extending from page 2, line 33 to page 12, line 14 of International PCT Patent Application Publication No. WO 2016/120380 can be used to formulate compositions containing secretin, in place of a GLP-1 agonist, for oral deliver to mammals to treat FD and/or to reduce the symptoms of FD as described herein.

In some cases, a pharmaceutical composition can be formulated to include secretin (or one or more secretin receptor agonists, one or more positive allosteric modulators of secretin, one or more neprilysin inhibitors, or a combination thereof) for intravenous administration to a mammal (e.g., a human) having FD. The intravenous administration of secretin can be accomplished by attaching a winged infusion set, also known as a “butterfly needle,” to a syringe containing a composition that includes secretin and inserting the needle into a vein in the arm of a human being treated. The composition containing secretin can then be pushed through the syringe into the bloodstream of the human.

In some cases, a pharmaceutical composition can be formulated to include secretin (or one or more secretin receptor agonists, one or more positive allosteric modulators of secretin, one or more neprilysin inhibitors, or a combination thereof) for topical administration to a mammal (e.g., a human) having FD. Any appropriate method can be used to formulate secretin into a composition for topical delivery to a mammal (e.g., a human) having FD. For example, the methods described elsewhere (see, e.g., U.S. Patent Application Publication No. 2013/0085105, U.S. Pat. No. 6,432,383, or U.S. Pat. No. 5,023,252), which describe formulating other polypeptide compositions for topical delivery, can be used to formulate secretin into a composition for topical delivery.

A composition containing secretin (or one or more secretin receptor agonists, one or more positive allosteric modulators of secretin, one or more neprilysin inhibitors, or a combination thereof) for treating FD as described herein can be administered at to a mammal in an effective amount, at an effective frequency, and for an effective duration. Effective doses can vary depending on the severity of the FD, the route of administration, the age and general health condition of the subject, excipient usage, the possibility of co-usage with other therapeutic treatments such as use of acid suppression agents, and the judgment of the treating physician.

An effective amount of a composition containing secretin (or one or more secretin receptor agonists, one or more positive allosteric modulators of secretin, one or more neprilysin inhibitors, or a combination thereof) can be any amount that reduces the severity of a symptom of FD without producing significant toxicity to the mammal. For example, an effective amount of secretin can be from about 0.2 mcg/kg to about 100 mcg/kg (e.g., from about 0.2 mcg/kg to about 0.4 mcg/kg over one minute). The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the FD may require an increase or decrease in the actual effective amount administered.

The frequency of administration can be any frequency that reduces the severity of a symptom of FD without producing significant toxicity to the mammal. For example, the frequency of administration can be from about once a day to about three times a day. The frequency of administration can remain constant or can be variable during the duration of treatment. In some cases, treatment with secretin (or one or more secretin receptor agonists, one or more positive allosteric modulators of secretin, one or more neprilysin inhibitors, or a combination thereof) can on an as needed basis. As with the effective amount, various factors can influence the actual frequency of administration used for a particular application. For example, the effective amount, duration of treatment, use of multiple treatment agents, route of administration, and severity of the FD may require an increase or decrease in administration frequency.

An effective duration for administering a composition containing secretin (or one or more secretin receptor agonists, one or more positive allosteric modulators of secretin, one or more neprilysin inhibitors, or a combination thereof) can be any duration that reduces the severity of a symptom of FD without producing significant toxicity to the mammal. For example, the effective duration can vary from several days to several weeks, months, or years. In some cases, the effective duration for the treatment of FD can range in duration from about one month to about 10 years. Multiple factors can influence the actual effective duration used for a particular treatment. For example, an effective duration can vary with the frequency of administration, effective amount, use of multiple treatment agents, route of administration, and severity of the FD being treated.

In some cases, a course of treatment and the severity of one or more symptoms of the FD being treated can be monitored. Any appropriate method can be used to determine whether or not the severity of a symptom of FD is reduced. For example, the severity of a symptom of FD can be assessed using a gastric accommodation scan or a gastric emptying study at different time points.

The invention will be further described in the following examples, which do not limit the scope of the invention described in the claims.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating functional dyspepsia in a mammal, wherein said method comprises administering a composition comprising secretin to a mammal identified as having functional dyspepsia, wherein a symptom of said functional dyspepsia is reduced.

2. The method of claim 1, wherein said mammal is a human.

3. The method of claim 1, wherein said secretin is human secretin.

4. The method of claim 1, wherein the sole active ingredient of said composition is secretin.

5. The method of claim 1, wherein said composition comprises a positive allosteric modulator of secretin.

6. The method of claim 1, wherein said composition comprises a neprilysin inhibitor.

7. The method of claim 6, wherein said neprilysin inhibitor is sacubitril or sacubitrilat.

8. The method of claim 1, wherein said composition is administered intravenously or orally to said mammal.

9. A method for treating functional dyspepsia in a mammal, wherein said method comprises administering a composition comprising neprilysin inhibitor to a mammal identified as having functional dyspepsia, wherein a symptom of said functional dyspepsia is reduced.

10. The method of claim 9, wherein said mammal is a human.

11. The method of claim 9, wherein said neprilysin inhibitor is sacubitril or sacubitrilat.

12. The method of claim 9, wherein the sole active ingredient of said composition is said neprilysin inhibitor.

13. The method of claim 9, wherein said composition comprises secretin.

14. The method of claim 13, wherein said secretin is a human secretin.

15. The method of claim 9, wherein said composition comprises a positive allosteric modulator of secretin.

16. The method of claim 9, wherein said composition is administered intravenously or orally to said mammal.
